(19) United States
(12) Patent Application Publication
(10) Pub. No.: US 2008/0311542 A1
(43) Pub. Date: Dec. 18, 2008

(54) Intra-Oral Distraction Device

(76) Inventors: Grishma Rana, Old Bridge, NJ (US); Dorothy White, Fanwood, NJ (US)

Correspondence Address: GREENBERG TRAURIG, LLP, 200 PARK AVE., P.O. BOX 677, FLORHAM PARK, NJ, 07932, US

(21) Appl. No.: 11/910,485

(22) PCT Filed: Apr. 6, 2006
(86) PCT No.: PCT/US06/13202

§ 371 Date: Aug. 4, 2008

Related U.S. Application Data
(60) Provisional application No. 60/668,818, filed on Apr. 6, 2005.

(51) Int. Cl. A61C 5/00; A61B 17/58

(52) U.S. Cl. 433/140; 606/90

(57) ABSTRACT

A distraction device that allows for an improved use of the existing process of distraction osteogenesis. The distraction device facilitates the displacement of a healthy portion of bone to a deficient area to enable bone and soft tissue growth in a fracture gap and allows for both vertical and angular bone regeneration.

### CROSS-REFERENCE TO RELATED APPLICATION

This application claims the benefit of U.S. Provisional Application No. 60/668,818, filed Apr. 6, 2005. U.S. Provisional Application No. 60/668,818 is hereby incorporated herein by reference.

### FIELD OF THE INVENTION

The present invention relates to a device for regenerating bone in a patient, particularly for use in a distraction osteogenesis method. Specifically, the present invention relates to an improved device that facilitates angled and vertical bone regeneration in the oral cavity, allowing for bone growth in a direction that is not necessarily vertical to the device.

### BACKGROUND OF THE INVENTION

During the 1950's, Professor Gavril Ilizaroz revealed that controlled, mechanically applied tension stress on fractured bone produces successful regeneration of bone and soft tissue, a technique known today as distraction osteogenesis. Initially used for the treatment of fractures and non-unions, his methods have proven successful for limb lengthening and bone transportation.

Today, distraction osteogenesis is used to lengthen long bones, correct bone deformities, correct maxillofacial abnormalities, and treat periodontal bone deficiency. Generally, distraction osteogenesis involves making small bone cuts at precise positions and applying forces across these cuts using a distraction device to induce and direct normal bone formation.

More specifically, a typical procedure for distraction osteogenesis involves the following four sequentially performed stages: osteotomy, latency, distraction, and consolidation. The first stage, osteotomy, involves cutting a section of a bone requiring treatment into two pieces, known as a controlled fracture, thus initiating the fracture's natural healing process. Fracture healing is a process whereby fibrous deformable tissue forms and is gradually replaced by non-deformable tissue (i.e., ossification).

The second stage, latency, is a “rest” period (e.g., approximately 5-7 days), during which healing of the fracture proceeds until a blood clot is formed. Upon formation of the blood clot, the distraction stage begins. Distraction is a process wherein bony fragments or joint surfaces of a limb are separated or otherwise spread by extension. During distraction, a “gap” is created at the site of the osteotomy. Following formation of the appropriately sized gap, a consolidation stage, or period of healing, occurs.

During the distraction stage, conventional devices are employed to control and direct the bone distraction. However, conventional distraction devices for intra-oral distraction facilitate bone regeneration in only a vertical direction. Because bone geometries vary from patient to patient and include overlaps and non-vertical angles, vertical regeneration alone does not provide the necessary treatment option for many patients.

Accordingly, there is a need for a distraction device that allows for improved regeneration of bone, particularly in the oral cavity.

### SUMMARY OF THE INVENTION

The present invention relates to a distraction device and a method for facilitating angled and vertical bone regeneration in the oral cavity. The device is particularly suited for use by oral surgeons, periodontists, plastic surgeons, and maxilla specialists in the treatment of traumatic injury, cancer, tumor ablation, or growth disharmony, such as those affecting the temporo-mandibular joint (TMJ). The distraction device facilitates the displacement of a healthy portion of bone to a deficient area to allow for bone regeneration and soft tissue growth. Advantageously, the distraction device allows for both vertical and angular bone regeneration by facilitating both vertical and angular distraction.

The distraction device according to an embodiment of the present invention may be used to regenerate bone tissue in deficient areas of a patient's upper and/or lower jaw bones (i.e., the maxilla and/or mandible bones) to allow for subsequent dental implantation. According to an aspect of the present invention, the distraction device includes multi-tooth capabilities based on a patient's needs. One having ordinary skill in the art will appreciate that the distraction device may be used for vertical and angular bone regeneration in other areas of the body and is not limited to intra-oral use.

According to an embodiment of the present invention, the distraction device includes a threaded rod with a connecting wedge housing at one end, a threaded nut for engaging with the threaded rod, two lower transport plates for positioning on either side of a lower portion of the threaded rod, an upper transport plate for securing an upper portion of the threaded rod, and one or more connecting wedges adapted to fit in the distraction device.

Each connecting wedge includes a main body and a connecting-wedge extension extending from each side of the main body. The main body of the connecting wedge is offset relative to the connecting-wedge extensions at an angle, referred to as an “offset angle.” The term “wedge” is intended to include, but is not limited to, any structure, device, piece, component, etc. having any suitable shape or size such that it is adapted to provide an offset angle as described herein. As such, the term “wedge” may not be limited to any conventional definition, particular shape, and/or geometric configuration.

In operation, the two lower transport plates are fixed to a healthy portion of bone or tissue adjacent an area of bone deficiency according to a technique known in the art. An upper transport plate is secured to the site of an osteotomy with a securing mechanism, such as for example standard bone screws. The upper transport plate includes a sleeve through which the threaded rod is passed, thereby releasably coupling the upper transport plate and the threaded rod. The threaded nut is engaged on the threaded rod and may be moved to shift the upper transport plate and adjust a vertical component of the distraction (i.e., the vertical distraction).

The main body of the connecting wedge is fitted within the connecting wedge housing of the threaded rod. With the main body securely attached to the threaded rod, the connecting wedge extensions are engaged within the lower transport plates, and the threaded rod is stabilized by the lower transport plates. Further, when the threaded rod, the connecting wedge, and the lower transport plates are assembled, the threaded rod extends at an angle that is offset relative to the lower transport plates. That is, the threaded rod and the lower transport plates form an angular relationship that is not necessarily parallel or perpendicular. The angular arrangement of the threaded rod relative to the lower transport plates allows for angular distraction and angular bone regeneration.

In another embodiment of the present invention, the distraction device includes a threaded rod engaged within a correspondingly threaded hub. The distraction device also includes a shaft that passes horizontally through the hub, wherein each end of the shaft extending out of the sides of the hub are secured by a lower transport plate. The threaded rod, the hub, and the shaft combine to form a setscrew. When rotated in a clockwise direction, the threaded rod moves in a downward direction within the hub and into contact with the shaft. When sufficient contact between the threaded rod and the shaft is established, the threaded rod and the hub are prevented from rotating about the shaft, and the distraction device is in a locked position.

Correspondingly, when the threaded rod is turned in a counter-clockwise direction, the threaded rod moves in an upward direction and out of contact with the shaft, placing the distraction device in an unlocked position. In the unlocked position, the threaded rod and hub are free to rotate about the shaft and may be positioned at an angle relative to the lower transport plates. When the desired angle of the threaded rod (i.e., the desired angle of bone regeneration) has been assigned, the angle may be set by turning the threaded rod in the clockwise direction to place the distraction device in the locked position. Advantageously, the distraction device allows for the angle of regeneration to be adjusted at any time during the distraction procedure.

### BRIEF DESCRIPTION OF THE DRAWINGS

The present invention will be more readily understood from the detailed description of the preferred embodiment(s) presented below considered in conjunction with the attached drawings, of which:

FIG. 1 is an isometric view of a distraction device, according to an embodiment of the present invention;

FIG. 2 is a rear view of a distraction device, according to an embodiment of the present invention;

FIG. 3 is a side view of a distraction device, according to an embodiment of the present invention;

FIG. 4 is an isometric view of a connecting wedge, according to an embodiment of the present invention;

FIG. 5 is a side view of a connecting wedge, according to an embodiment of the present invention;

FIG. 6 is an exploded view of a distraction device, according to an embodiment of the present invention;

FIG. 7 depicts an exemplary connecting wedge having an offset angle of 0° and a distraction device including the exemplary connecting wedge, according to an embodiment of the present invention;

FIG. 8 depicts an exemplary connecting wedge having an offset angle of 10° and a distraction device including the exemplary connecting wedge, according to an embodiment of the present invention;

FIG. 9 is an isometric illustration of a distraction device, according to an embodiment of the present invention;

FIG. 10 depicts an exemplary hub, according to an embodiment of the present invention; and

FIG. 11 depicts an exemplary shaft, according to an embodiment of the present invention.

It is to be understood that the attached drawings are for the purpose of illustrating concepts of the present invention and may not be to scale.

### DETAILED DESCRIPTION OF THE INVENTION

The present invention relates to a distraction device for facilitating both vertical and angular bone regeneration, particularly in the oral cavity.

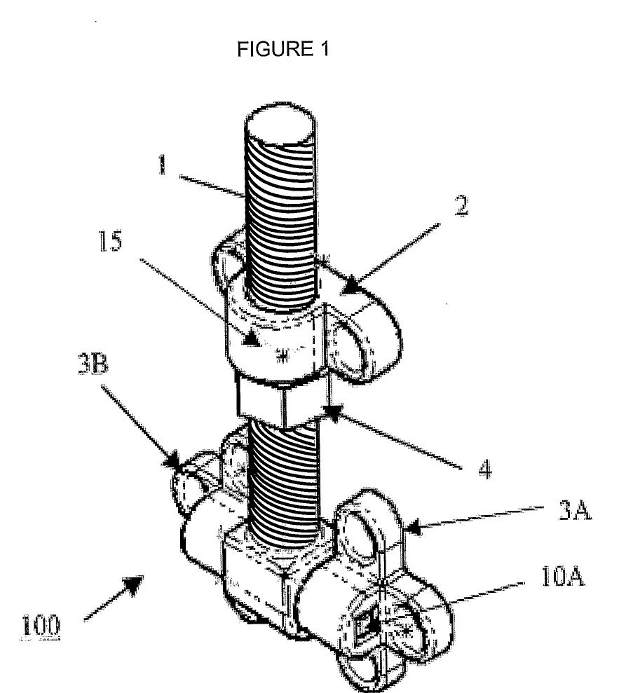

FIGS. 1, 2, 3, and 6 depict an exemplary distraction device 100 according to an embodiment of the present invention. The distraction device 100 includes a threaded rod 1, a threaded nut 4 disposed on the threaded rod 1, an upper transport plate 2, one or more lower transport plates 3A, 3B, and a connecting wedge 5. At one end of the threaded rod 1 is a connecting wedge housing 11 that includes an opening 12 shaped and sized to fit the connecting wedge 5, as described in detail below. In a preferred embodiment, the threaded rod 1 is composed of M0.5×3 standard isometric threads.

According to an embodiment of the present invention, the one or more lower transport plates 3A, 3B are adapted to be secured or attached to a healthy portion of a patient's bone and/or tissue surrounding the area of bone deficiency, referred to as a target area. For example, if the mandible is the target area, the one or more lower transport plates 3A, 3B are fixed below the target bone. In another example, if the maxilla is the target area, the one or more lower transport plates are fixed above the target area. One having ordinary skill in the art will appreciate that the lower transport plates 3A, 3B may be secured proximal to the target area by any suitable fastening mechanism or method, including, but not limited to, a screw assembly.

According to a preferred embodiment, two lower transport plates 3A, 3B are employed. Although the distraction device 100 is described as preferably including two lower transport plates 3A, 3B, one having ordinary skill in the art will appreciate that any number of lower transport plates may be employed, with an important consideration being that the lower portion of the distraction device 100 is adequately secured in place.

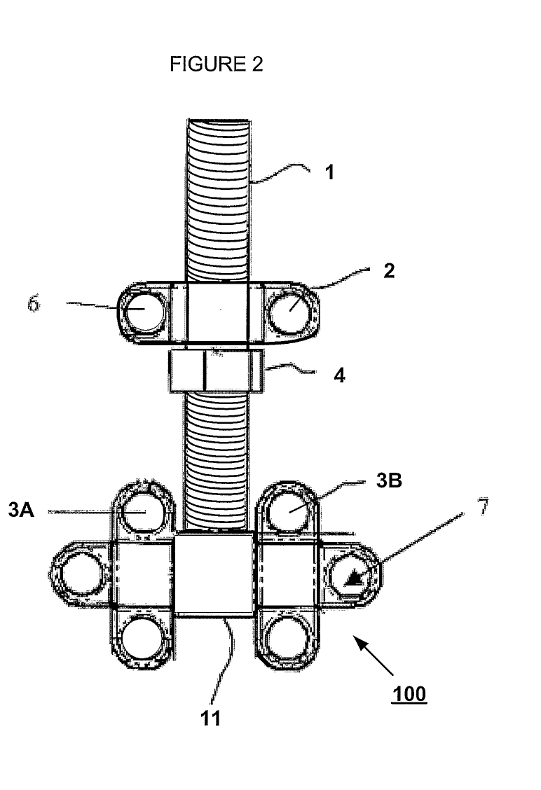

The lower transport plates 3A, 3B each include one or more holes 7, as shown in FIG. 2. Each hole 7 is adapted to accept a screw or similar securing mechanism for affixing the lower transport plates 3A, 3B to the area adjacent the target area. Preferably, the lower transport plates 3A, 3B are arranged relatively coplanar to each other.

The upper transport plate 2 is fixed at or near a target area (e.g., the site of an osteotomy) by one or more bone screws passing through one or more holes 6 of the upper transport plate 2, as shown in FIG. 2. The upper transport plate 2 includes a sleeve 15 with an opening sized and shaped to surround the threaded rod 1. As shown in FIGS. 1, 2, and 6, the threaded rod 1 is releasably coupled to the upper transport plate 2, by passing the threaded rod 1 through the sleeve 15 of the upper transport plate 2, such that the threaded rod 1 acts as a transport vector for the bone distraction. One having ordinary skill in the art will appreciate that the threaded rod 1 and the upper transport plate 2 may be releasably coupled to one another according to any suitable means or mechanism. One having ordinary skill in the art will appreciate that the upper transport plate 2 and the lower transport plates 3A, 3B may be manufactured using any known method, such as, for example, a machining method, a die casting method, or a Computer Numerical Control (CNC) method.

Disposed on the threaded rod 1 is the threaded nut 4. The threaded nut 4 is threadably engaged on the threaded rod 1 such that the threaded nut 4 is able to move axially along the threaded rod 1 upon the application of a force. To adjust an amount of vertical distraction provided by the distraction device 100, a user turns the threaded nut 4 thus causing the threaded nut 4 to move into contact with the upper transport plate 2 and shift the upper transport plate 2 in the desired direction by the desired amount. Because the interior surface of the sleeve 15 is not threaded, the osteotomy moves longitudinally, in parallel with the threaded rod 1.

In a preferred embodiment, the threaded nut 4 may be turned using a suitable tool, such as for example a small hexagonal wrench. In an exemplary configuration, the threaded rod 1 includes threads having a pitch of approximately 0.5 mm. According to this exemplary arrangement, the user may turn the threaded nut 4 one complete turn twice a day in order to shift the upper transport plate 2 a total distance of approximately 1 mm a day. Although the threaded nut 4 and the threaded rod 1 are described as engaged by a threaded connection, alternative arrangements for disposing the threaded nut 4 on the threaded rod 1 are contemplated, such that the threaded nut 4 is movable along the threaded rod 1.

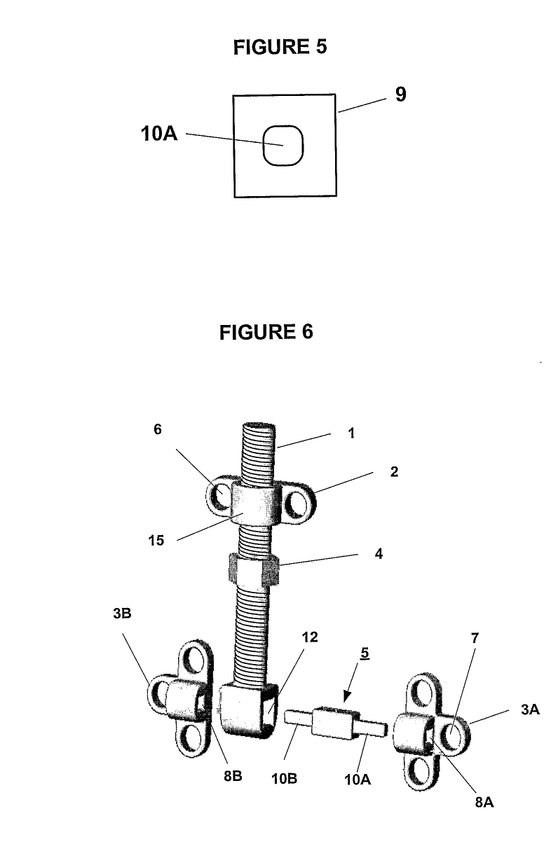

FIGS. 4 and 5 depict an exemplary connecting wedge 5 according to an embodiment of the present invention. The connecting wedge 5 attaches the lower transport plates 3A, 3B to the threaded rod 1, and also provides a mechanism for setting the angle of the threaded rod 1 relative to the lower transport plates 3A, 3B. According to an embodiment of the present invention, the connecting wedge 5 includes a main body 9 and two connecting wedge extensions 10A, 10B extending from opposing sides of the main body 9. Preferably, both the main body 9 and the connecting wedge extensions 10A, 10B have a rectangular shape, as shown in FIG. 4. One having ordinary skill in the art will appreciate that the main body 9 and the connecting wedge extensions 10A, 10B may be integrally formed as a single piece.

The main body 9 of the connecting wedge 5 is offset relative to the connecting wedge extensions 10A, 10B at a particular angle, referred to as an “offset angle,” graphically represented by the symbol “θ” in FIG. 8. One having ordinary skill in the art will appreciate that the connecting wedge 5 may be configured to have any desired offset angle. One having ordinary skill in the art will appreciate that each connecting wedge 5 may be configured such that the relative arrangement of the main body 9 and the connecting wedge extensions 10A, 10B is adjustable (i.e., the position of the main body 9 and/or the connecting wedge extensions 10A, 10B may be made changed to set a new offset angle.) For example, the connecting wedge extensions 10A, 10B may be adapted to rotate relative to the main body 9, to adjust the offset angle provided by the connecting wedge 5.

An exemplary connecting wedge 5 having an offset angle of 10° (i.e., the main body 9 is offset relative to the connecting wedge extensions 10A, 10B at an angle of 10°) is shown in FIG. 8.

One having ordinary skill in the art will appreciate that the distraction device 100 according to an embodiment of the present invention may be adapted to fit any one of a collection of interchangeable connecting wedges 5, each having a particular offset angle. Further, it is to be appreciated that the main body 9 may be any shape or size, with an important consideration being that the main body 9 fits securely within the opening 12 of the connecting wedge housing 11 and is adapted for arrangement relative to the connecting wedge extensions 10A, 10B to produce an offset angle.

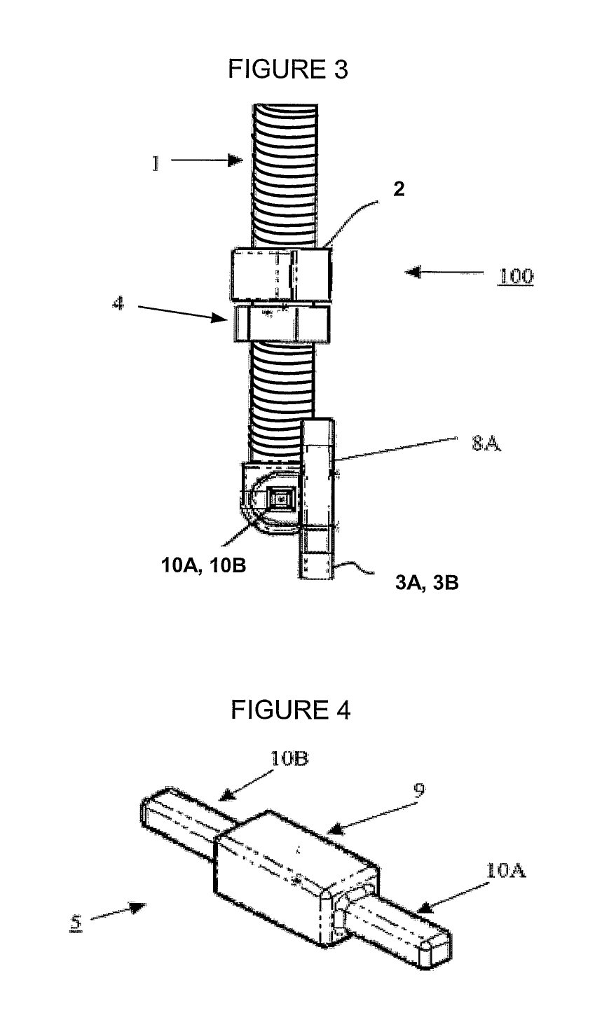

Each lower transport plate 3A, 3B includes an opening 8A, 8B that extends perpendicularly to the surface of the target area, as shown in FIGS. 3 and 6. The opening 8A, 8B are adapted to accept and secure the connecting wedge extension 10A, 10B. The openings 8A, 8B of the lower transport plates 3A, 3B have the same shape as the connecting wedge extensions 10A, 10B. Preferably, the openings 8A, 8B and the connecting wedge extensions 10A, 10B are square-shaped.

The distraction device 100 is assembled by fitting the main body 9 into the opening 12 of the connecting wedge housing 11 and engaging the connecting wedge extensions 10A, 10B within the openings 8A, 8B of the lower transport plates 3A, 3B, respectively, thus holding the threaded rod 1 and the lower transport plates 3A, 3B together. Further, an upper portion of the threaded rod 1 is secured by the upper transport plate 2, as described above. Advantageously, the distraction device 100 is stabilized by the lower transport plates 3A, 3B and the surrounding soft tissue of the region adjacent the target area, thus preventing any separation of the components of the distraction device 100 while in situ.

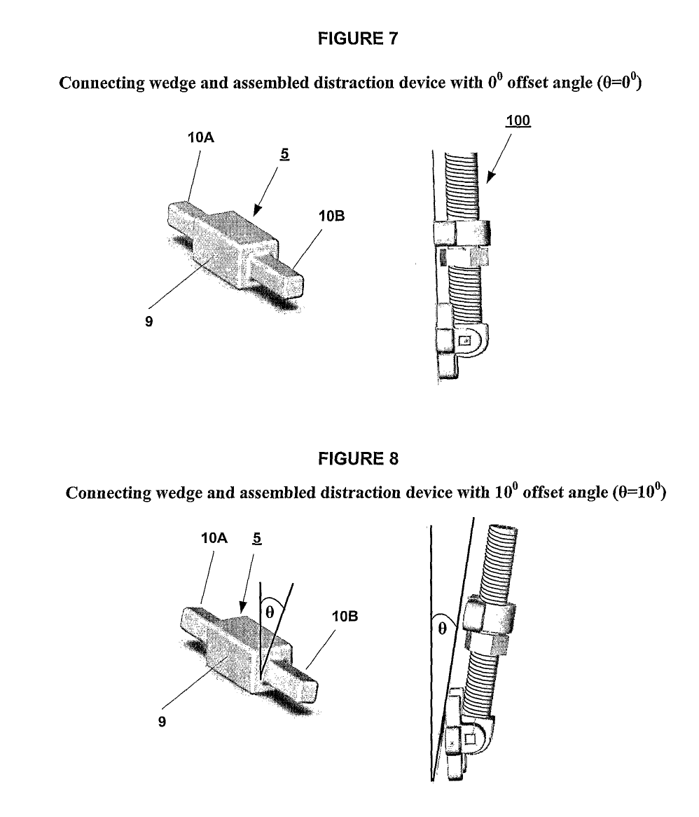

The offset angle of the connecting wedge 5 dictates the angle at which the threaded rod 1 extends relative to the lower transport housings 3A, 3B, and thus provides the angle of bone regeneration provided by the distraction device 100. FIG. 7 depicts an exemplary connecting wedge 5 with an offset angle θ of approximately 0° (i.e., the main body 9 is not offset relative to the connecting wedge extensions 10A, 10B). Accordingly, when the connecting wedge 5 depicted in FIG. 7 is loaded into the distraction device 100, there is an approximately 0° offset angle between the lower transport plates 3A, 3B and the threaded rod 1. Thus, the distraction device 100 according to this exemplary arrangement provides an approximately 0° angle of angular bone regeneration.

FIG. 8 depicts an exemplary connecting wedge 5 with an offset angle θ of 10°. Accordingly, when the connecting wedge 5 shown in FIG. 8 is loaded into the distraction device 100, the threaded rod 1 extends at a 10° angle relative to the lower transport plates 3A, 3B. Thus, the distraction device 100 according to this exemplary arrangement facilitates bone regeneration at a 10° angle.

Advantageously, each connecting wedge 5 may be configured at a different offset angle, to allow a user to select from a wide range of angles for angular bone regeneration, depending on the individual patient's needs. The distraction device 100 may be fitted with any one of a collection of connecting wedges 5 depending on the angle of angular bone regeneration desired.

One having ordinary skill in the art will appreciate that the distraction device 100 may be composed of any suitable material, such as for example titanium or surgical-grade stainless steel. Preferably, the distraction device 100 may be fabricated using known CNC technology.

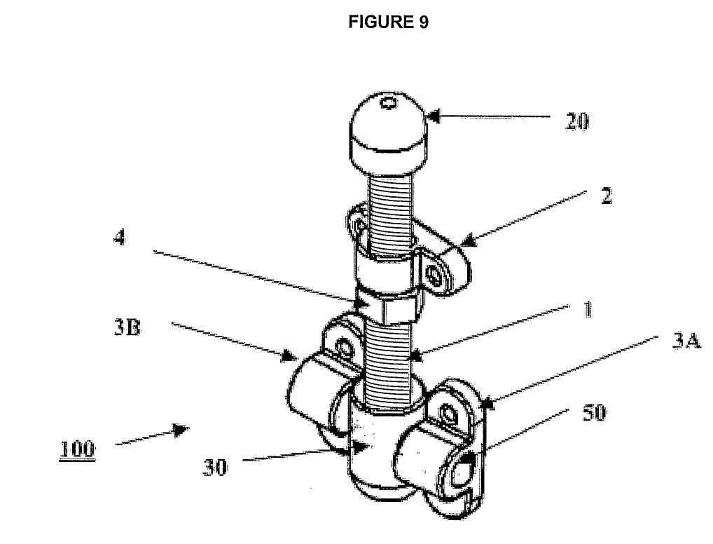

FIG. 9 illustrates a distraction device 100 according to another embodiment of the present invention. As shown in FIG. 9, the distraction device 100 includes an upper transport plate 2 adapted to be secured the site of an osteotomy. As described above, the upper transport plate 2 includes a sleeve 15 with an opening sized and shaped to surround the threaded rod 1.

According to this embodiment of the present invention, the distraction device 100 includes a threaded nut 4 engaged on a threaded rod 1, as described in detail above. As described above, the threaded nut 4 may be turned to shift the upper transport plate 2 to allow for vertical adjustment of the distraction device 100. As such, the threaded rod 1 acts as a transport vector for bone distraction. As described above, two lower transport plates 3A, 3B are secured to a healthy portion of the patient's bone or tissue proximal to the target area. The lower transport plates 3A, 3B each include a sleeve adapted to accept and secure the ends of a shaft 50.

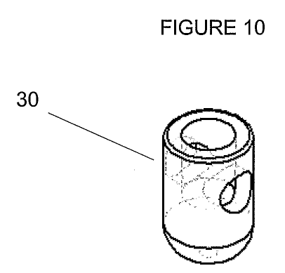

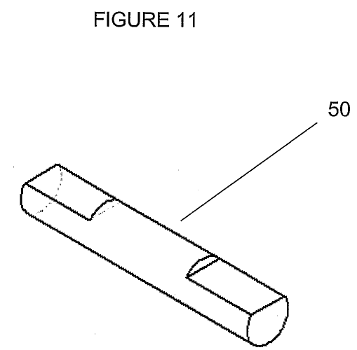

The distraction device 100 includes a hub 30 positioned between the lower transport plates 3A, 3B. The hub 30 includes an opening in its top and a threaded interior surface for insertion and engagement of the threaded rod 1, as shown in FIG. 10. The hub 30 also includes an opening on each side that aligns with the openings of the lower transport plates 3A, 3B to allow for insertion of the shaft 50, shown in FIG. 11. When assembled, the center portion of the shaft 50 is arranged within the hub 30 and the ends of the shaft 50 are held on either side by the lower transport plates 3A, 3B, such that the threaded rod 1 and the shaft 50 are substantially perpendicular to each other, as shown in FIG. 9. The threaded rod 1, the hub 30, and the shaft 50 combine to form a setscrew for adjustment of the angle of the threaded rod 1 relative to the lower transport plates 3A, 3B, which in turn dictates the distraction angle.

A knob 20, or other equivalent structure, is positioned at an end of the threaded rod 1 and is used to adjust the position of the threaded rod 1. In operation, the knob 20 may be turned in a clockwise direction to cause the threaded rod 1 to move downwardly into the hub 30 until the threaded rod 1 makes contact with the shaft 50. When the threaded rod 1 is moved into sufficient contact with the shaft 50, the hub 30 and threaded rod 1 are prevented from rotating about the shaft 50, thus placing the distraction device 100 in a locked position at the desired distraction angle.

Turning the knob 20 of the threaded rod 1 in a counter-clockwise direction moves the threaded rod 1 out of contact with the shaft 50, thus placing the device 100 in an unlocked position. In the unlocked position, the threaded rod 1 and hub 30 may be rotated about the shaft 50 to a desired distraction angle. As described above, while maintaining the desired angle, the user may turn the knob 20 in a clockwise direction causing the threaded rod 1 to rotate into contact with the shaft 50, thus setting the distraction device 100 in a locked position at the desired distraction angle. Advantageously, this embodiment of the present invention allows for in situ adjustment of the angle of regeneration provided by the distraction device 100. The distraction device 100 according to this embodiment of the present invention allows for the angle of regeneration to be adjusted at any time during the distraction procedure.

Although the present invention has been described in considerable detail with reference to certain preferred embodiments and version, other versions and embodiments are possible. Therefore, the scope of the present invention is not limited to the description of the versions and embodiments expressly disclosed herein. The references and disclosure provided in the ‘Background of the Invention’ section are not admitted to be prior art with respect to the disclosure provided in the present application.

What is claimed is:

1. A distraction device for facilitating bone regeneration comprising:
an upper transport plate adapted to be secured proximal to a target area;
at least one lower transport plate adapted to be secured proximal to the target area;
a threaded rod releasably coupled with the upper transport plate and the at least one lower transport plate, wherein the threaded rod includes a connecting wedge housing;
a threaded nut engaged on the threaded rod, such that the threaded nut translates axially along the threaded rod and shifts the upper transport plate to affect vertical distraction of the target area; and
a connecting wedge loaded in the connecting wedge housing of the threaded rod which couples the threaded rod and the lower transport plate at an offset angle to affect angular distraction of the target area.

2. The device of claim 1, wherein the upper transport plate includes a sleeve adapted to pass and hold the threaded rod.

3. The device of claim 1, wherein the upper transport plate include one or more holes for accepting a securing mechanism to secure the upper transport plate in place.

4. The device of claim 1, wherein the at least one lower transport plate includes one or more holes for accepting a securing mechanism to secure the at least one lower transport plate in place.

5. The device of claim 3, wherein the securing mechanism is a bone screw.

6. The device of claim 1, wherein the threaded nut is moved axially along the threaded rod by a tool.

7. The device of claim 1, wherein the connecting wedge includes a main body and two connecting wedge extensions, wherein the main body and the two connecting wedge extensions are arranged at the offset angle.

8. The device of claim 1, wherein each of the at least one lower transport plates includes an opening adapted to secure each connecting wedge extension.

9. The device of claim 1, wherein the connecting wedge housing is adapted to secure the main body of the connecting wedge.

10. The device of claim 1, wherein the at least one lower transport plate is attached proximal to the target area.

11. The device of claim 1, wherein the target area is in the oral cavity.

12. An intra-oral distraction device for facilitating bone regeneration comprising:
an upper transport plate adapted to be secured proximal to a target area;
at least one lower transport plate adapted to be secured proximal to the target area;
a threaded rod coupled with the upper transport plate;
a hub releasably coupled to the at least one lower transport plate, wherein the hub is adapted for threaded engagement with the threaded rod;
a threaded nut engaged on the threaded rod, such that the threaded nut translates axially along the threaded rod and shifts the upper transport plate to affect vertical distraction of the target area;
a shaft passing through the at least one lower transport plate and the hub, wherein the threaded rod and hub are rotatable about the shaft to a desired distraction angle, and wherein the threaded rod may be translated into contact with the shaft to set the device in a locked position at the desired distraction angle.

13. The device of claim 12, wherein the threaded rod includes a knob at one end for rotating the threaded rod within the hub.

14. A kit comprising:
a threaded rod and nut assembly including a connecting wedge housing;
a plurality of transport plates adapted to be secured proximal to a target area; and
a plurality of interchangeable connecting wedges adapted for loading into the connecting wedge housing, wherein connecting wedge has a unique offset angle.

15. The kit of claim 14, further comprising at least one securing mechanism for securing the upper and lower transport plates proximal to the target area.
